(19) United States
(12) Patent Application Publication
(10) Pub. No.: US 2025/0328001 A1
(43) Pub. Date: Oct. 23, 2025

(54) END CAP FOR COHERENT FIBRE BUNDLE FOR ENABLING SELECTIVE PLANE ILLUMINATION MICROSCOPY

(71) Applicant: Heriot-Watt University, Edinburgh (GB)

(72) Inventors: Robert R. Thomson, Edinburgh (GB); Pablo Roldan-Varona, Edinburgh (GB); Michael G. Tanner, Edinburgh (GB); Helen Elizabeth Parker, Edinburgh (GB); Calum Ross, Edinburgh (GB)

(73) Assignee: Heriot-Watt University, Edinburgh, JJ (GB)

(21) Appl. No.: 18/857,768

(22) PCT Filed: Apr. 20, 2023
(86) PCT No.: PCT/GB2023/051040

§ 371 Date: Oct. 17, 2024

(30) Foreign Application Priority Data
Apr. 21, 2022 (GB) 2205826.7

(51) Int. Cl. G02B 21/36; G01N 21/64; G01N 21/65; G02B 21/00; G02B 23/16; G02B 23/24

(52) U.S. Cl. CPC G02B 21/361; G02B 21/0076; G02B 23/16; G02B 23/2469; G01N 21/6458; G01N 21/65

(57) ABSTRACT

An end cap (12) for a coherent fibre bundle, CFB, (6) for enabling selective plane illumination microscopy, SPIM, is disclosed, wherein the end cap (12) comprises one or more CFB (6) alignment features for aligning the end cap (12) relative to a distal end of a CFB (6), a sample space (44) for receiving a sample or material to be imaged, and a peripheral reflector (50) arranged at least part way around the sample space (44), wherein the sample space (44) extends from a front side (46) of the end cap (12), and wherein the end cap (12) is configured so that, when the end cap (12) is aligned relative to the distal end of the CFB (6), the peripheral reflector (50) re-directs excitation light output from a plurality of outer optical cores of the CFB (6) so that the re-directed excitation light propagates at least part way across the sample space (44) in front of an end face of the CFB (6) for the excitation of the sample or material in the sample space (44) and the generation of fluorescence therein and so that at least a portion of the fluorescence is coupled into a plurality of inner optical cores of the CFB (6). The end cap (12) may be used for enabling selective plane illumination microscopy (SPIM) and, in particular though not exclusively, for enabling SPIM for clinical endomicroscopy.

### FIELD

The present disclosure relates to an end cap for a coherent fibre bundle (CFB) for enabling selective plane illumination microscopy (SPIM) and, in particular though not exclusively, for enabling SPIM for clinical endomicroscopy.

### BACKGROUND

It is known to use coherent fibre bundles (CFBs) to transmit images for in vivo medical microscopy of various organs of the body in a minimally-invasive way due to their narrow diameter (typically <1 mm), flexibility, and chemical and bio-inertness. For example, it is known to use CFBs for endomicroscopy of urinary, gastrointestinal, and respiratory organs.

However, use of a CFB in a wide-field modality may result in unwanted background fluorescence being observed in images acquired through the fibre. This background originates from out-of-focus fluorescence which is emitted outside of the image plane and may degrade the quality and/or contrast of images acquired through the CFB.

Moreover, it is known to use CFBs formed from highly doped silica. However, such silica CFBs can be expensive. Consequently, it is known to use polymer CFBs as these can have higher core-cladding refractive index contrasts, higher diameter CFBs with larger fields of view, and are inherently fabricated using lower cost materials and facilities than silica CFBs. However, polymer CFBs can generate a higher level of auto-fluorescence when pumped with near-UV or blue light than silica CFBs. This can be prohibitive for fluorescence imaging, particularly at shorter wavelengths, e.g., for imaging at wavelengths in the green region of the visible spectrum where many clinically relevant endogenous fluorophores fluoresce.

It is also known to perform selective plane illumination microscopy (SPIM) through a CFB. However, known systems for performing SPIM through a CFB use a separate excitation fibre next to the CFB. Moreover, such known systems for performing SPIM through a CFB may rely upon the use of one or more additional optical components at the distal end of the excitation fibre and/or at the distal end of the CFB. For example, such known systems for performing SPIM through a CFB may include a GRIN lens at the distal end of the excitation fibre and/or a GRIN lens at the distal end of the CFB. Such known systems for performing SPIM through a CFB may use a microprism to generate the excitation light sheet for SPIM. Consequently, such known systems for performing SPIM through a CFB may be complex and cumbersome and may have a distal-end cross-section of several millimetres or more across, wherein much of this space is taken up by the additional optical components at the distal end of the excitation fibre. This may also reduce the field of the view. Consequently, use of such known systems for performing SPIM through a CFB may be prohibitive for SPIM for some endoscopic applications which require a smaller cross-section and/or a larger field of view.

It is also known to use structured illumination microscopy through a CFB, whereby illumination patterns are projected onto a sample to be imaged, to minimise out-of-focus fluorescence background. However, the use of structured illumination microscopy through a CFB may result in motion artefacts in the image of the sample. Moreover, known systems which use structured illumination microscopy through a CFB do not address the generation of any auto-fluorescence background in the CFB itself.

### SUMMARY

According to an aspect of the present disclosure there is provided an end cap for a coherent fibre bundle (CFB) for enabling selective plane illumination microscopy (SPIM), the end cap comprising:
one or more CFB alignment features for aligning the end cap relative to a distal end of a CFB;
a sample space for receiving a sample or material to be imaged, wherein the sample space extends from a front side of the end cap; and
a peripheral reflector arranged at least part way around the sample space,
wherein the end cap is configured so that, when the end cap is aligned relative to the distal end of the CFB, the peripheral reflector re-directs excitation light output from a plurality of outer optical cores of the CFB so that the re-directed excitation light propagates at least part way across the sample space in front of an end face of the CFB for the excitation of the sample or material in the sample space and the generation of fluorescence therein and so that at least a portion of the fluorescence is coupled into a plurality of inner optical cores of the CFB.

Such an end cap can be aligned and/or installed on the distal end of the CFB. The end cap may be configured to accept excitation light delivered through the outer optical cores and to re-direct the excitation light so as to form a light sheet which propagates at least part way across the sample space in front of the end face of the CFB and which may, for example, be generally parallel to the end face of the CFB. This may allow the CFB to be used for the SPIM of any sample or material located in the sample space.

Moreover, the fluorescence emitted from the excited sample or material is captured by the inner optical cores of the CFB and an image of the fluorescence is transmitted by the inner optical cores of the CFB back to an image sensor located at a proximal end of the CFB. Consequently, use of such an end cap means that the inner optical cores of the CFB are not excited by the excitation light thereby avoiding, or at least partially suppressing, the generation of any auto-fluorescence background in the inner optical cores of the CFB. This may improve image quality and/or image contrast, particularly when the end cap is used with polymer CFBs, which can generate a strong fibre auto-fluorescence background.

Use of such an end cap with a polymer CFB may be advantageous for endoscopy not least because polymer materials such as PMMA can be fabricated into CFBs which are more flexible and less fragile than CFBs formed from a glass material such as silica. For example, it has been found that a PMMA CFB with an outer diameter of 1.5 mm is flexible enough to be deployed down an endoscope, whereas a glass CFB of the same diameter would be too rigid to be deployed down an endoscope. Use of a CFB assembly comprising such an end cap fitted to a distal end of a polymer CFB may be particularly advantageous for robotic-assisted endoscopy where a flexible, non-fragile CFB assembly is required.

Installing the end cap on the distal end of the CFB may enable SPIM so that only a region of the sample or material in the sample space which is in close proximity to the end of the CFB, and which is therefore in-focus, is excited. Thus, use of the end cap may avoid, or at least partially suppress, the generation in the sample or material of any out-of-focus fluorescence background.

The end cap is also suitable for use with a single CFB avoiding any need for any additional optical fibres. The end cap also avoids any requirement for the use of additional optical components such as one or more GRIN lenses and/or prisms at the distal end of the CFB. For all of these reasons, use of the end cap enables a reduced footprint or volume compared with prior art fibre optic SPIM systems. In particular, when the end cap is installed on the distal end of the CFB, the resulting assembly may have a reduced diameter relative to known CFB assemblies for SPIM. Use of the end cap may also provide a larger field of view than known CFB assemblies for SPIM. These characteristics may make the end cap advantageous for clinical use cases.

Optionally, the peripheral reflector is annular or generally annular.

Optionally, the peripheral reflector defines a reflector surface which extends at least part way around the sample space.

Optionally, the end cap defines a longitudinal axis for alignment with a longitudinal axis of the CFB.

Optionally, the end cap is cylindrically symmetric about the longitudinal axis.

Optionally, a normal to the reflector surface extends along a direction having a radially outward component relative to the longitudinal axis of the end cap.

Optionally, the reflector surface has a linear profile when viewed on a longitudinal cross-section of the end cap which includes the longitudinal axis of the end cap.

Optionally, the reflector surface has a curved profile when viewed on a longitudinal cross-section of the end cap which includes the longitudinal axis of the end cap.

Optionally, the curved profile of the reflector surface is outwardly convex relative to the longitudinal axis of the end cap.

Optionally, the peripheral reflector comprises a reflective material or coating which is formed on, or disposed on, the reflector surface or which covers the reflector surface.

Optionally, the reflective material or coating comprises a metal.

Optionally, the reflective material or coating comprises silver.

Optionally, the end cap comprises a peripheral lens arranged at least part way around the sample space and located radially between the peripheral reflector and the sample space relative to the longitudinal axis, wherein the peripheral lens is configured to concentrate or focus the re-directed excitation light as the re-directed excitation light propagates at least part way across the sample space in front of the end face of the CFB towards the longitudinal axis of the end cap.

Optionally, the peripheral lens is configured to concentrate the re-directed excitation light on the longitudinal axis of the end cap or to bring the re-directed excitation light to a focus at the longitudinal axis of the end cap.

Optionally, the peripheral lens defines a lens profile which is inwardly convex relative to the longitudinal axis of the end cap.

Optionally, the peripheral lens at least partially defines the sample space.

Optionally, the peripheral lens is annular or generally annular.

Optionally, a normal to the reflector surface extends along a direction having a radially inward component relative to the longitudinal axis of the end cap.

Optionally, the curved profile of the reflector surface is inwardly concave relative to the longitudinal axis of the end cap.

Optionally, the one or more CFB alignment features comprise a rear space for receiving the distal end of the CFB, wherein the rear space extends from a rear side of the end cap.

Optionally, the end cap defines a passageway which extends from the rear side of the end cap to the front side of the end cap.

Optionally, the rear space comprises a wider rear section of the passageway such as a wider diameter rear section of the passageway.

Optionally, the sample space comprises a narrower front section of the passageway such as a narrower diameter front section of the passageway.

Optionally, the end cap is annular or generally annular.

Optionally, the sample space comprises a front recess defined in the front side of the end cap.

Optionally, the rear space comprises a rear recess defined in the rear side of the end cap.

Optionally, the end cap comprises an intervening portion which is configured to extend between the front recess and an end face of the CFB at the distal end of the CFB when the end cap is aligned relative to the distal end of the CFB. Such an intervening portion may separate the end face of the CFB from the sample or material in the front recess. Such an intervening portion may be configured to transmit at least a portion of the fluorescence to the plurality of inner optical cores of the CFB when the 25 end cap is aligned relative to the distal end of the CFB.

Optionally, the intervening portion extends between the front and rear recesses.

Optionally, the end cap is unitary.

Optionally, the end cap comprises first and second parts, wherein the first and second parts comprise one or more complementary alignment features for aligning the first and second parts relative to one another.

Optionally, the first part defines the one or more CFB alignment features, and the first and second parts together define the peripheral reflector.

Optionally, the first part defines the one or more CFB alignment features, and a reflector surface of the peripheral reflector, and the second part defines a reflective material or coating which covers the reflector surface when the first and second parts are aligned.

Optionally, the first part defines the one or more CFB alignment features and the second part defines the peripheral reflector.

Optionally, the end cap is configured for use with a coherent fibre bundle (CFB) which comprises, or is formed from, a polymer material such as PMMA.

Optionally, the end cap is configured for use with a coherent fibre bundle (CFB) which comprises, or is formed from, a glass material.

Optionally, the end cap is configured so that, when the end cap is aligned relative to the distal end of the CFB, the peripheral reflector re-directs excitation light output from the plurality of outer optical cores of the CFB so that the re-directed excitation light propagates at least part way across the sample space in front of the end face of the CFB for the excitation of the sample or material in the sample space and the generation of Raman scattered light therein and so that at least a portion of the Raman scattered light is coupled into the plurality of inner optical cores of the CFB. Such an end cap may be used for SPIM imaging of the Raman scattered light.

Optionally, the end cap comprises, or is formed from, a material which is transparent or substantially transparent to the excitation light.

Optionally, the end cap comprises, or is formed from, a material which is transparent or substantially transparent to the fluorescence.

Optionally, the end cap comprises, or is formed from, a material which is transparent or substantially transparent to the Raman scattered light.

Optionally, the end cap comprises, or is formed from, fused-silica.

Optionally, the end cap is formed by exposing one or more regions of a substrate to light and selectively chemically etching away a material of the substrate from the one or more exposed regions. Exposing the one or more regions of a substrate to light may increase the chemical etchability of the material of the substrate in the one or more exposed regions of the substrate.

Optionally, exposing the one or more regions of the substrate to light comprises using ultrafast laser inscription of the one or more regions of the substrate.

Optionally, the end cap is disposable.

According to an aspect of the present disclosure there is provided a coherent fibre bundle (CFB) assembly for SPIM, the CFB assembly comprising a coherent fibre bundle (CFB) and the end cap as described above attached to a distal end of the CFB.

Optionally, the CFB comprises, or is formed from, a polymer material such as PMMA.

Optionally, the CFB comprises, or is formed from, a glass material.

Optionally, the CFB assembly comprises an adhesive such as an epoxy between the end cap and the distal end of the CFB for attaching or securing the end cap to the distal end of the CFB.

Optionally, the CFB assembly comprises an outer sleeve around the end cap and the distal end of the CFB.

Optionally, the outer sleeve comprises, or is formed from, heat-shrink tubing.

It should be understood that any one or more of the features of any one of the foregoing aspects of the present disclosure may be combined with any one or more of the features of any of the other foregoing aspects of the present disclosure.

### BRIEF DESCRIPTION OF THE DRAWINGS

An end cap for a coherent fibre bundle (CFB) for enabling selective plane illumination microscopy (SPIM) and a CFB assembly comprising a CFB and the end cap will now be described by way of non-limiting example only with reference to the drawings of which:

FIG. 1 is a schematic of a fluorescence endomicroscopy system for lung imaging;

FIG. 2 is a schematic longitudinal cross-section of an end cap for a coherent fibre bundle (CFB) of the fluorescence endomicroscopy system of FIG. 1 for enabling selective plane illumination microscopy (SPIM);

FIG. 3A is a side view microscope image of a region of a fused silica substrate after femtosecond laser inscription of the fused silica substrate so as to define the geometry of the end cap, but before chemical etching of the inscribed regions of the fused silica substrate;

FIG. 3B is a plan view microscope image of the same region of the fused silica substrate as shown in FIG. 3A after femtosecond laser inscription of the fused silica substrate so as to define the geometry of the end cap, but before chemical etching of the inscribed regions of the fused silica substrate;

FIG. 3C is an image of a 10×10×1 mm substrate of fused silica in which twelve end cap parts are inscribed;

FIG. 4A is a side view microscope image of a first part of a fused silica end cap after a KOH etching process;

FIG. 4B is a plan view microscope image of a second part of the fused silica end cap after a KOH etching process;

FIG. 4C is a microscope image of the first part of the end cap of FIG. 4A in a slightly oblique position;

FIG. 5A is an image of the first part of the fused silica end cap of FIG. 4A and the second part of the fused silica end cap glued together to form the end cap;

FIG. 5B is an image of the fused silica end cap of FIG. 5A bonded to a PMMA CFB;

FIG. 5C is an image of a distal end of a CFB assembly including the fused silica end cap of FIG. 5A bonded to a PMMA CFB and including a protective heat-shrink tubing around the fused silica cap;

FIG. 6 is a schematic of the proximal end instrumentation of the fluorescence endomicroscopy system of FIG. 1;

FIGS. 7A and 7B show images obtained of a first tissue phantom without and with selective plan illumination respectively;

FIGS. 8A and 8B show images obtained of a second tissue phantom without and with selective plan illumination respectively;

FIGS. 9A and 9B show images obtained of a third tissue phantom without and with selective plan illumination respectively; and

FIG. 10 is a schematic longitudinal cross-section of an alternative end cap for a coherent fibre bundle (CFB) of the fluorescence endomicroscopy system of FIG. 1 for enabling selective plane illumination microscopy (SPIM).

### DETAILED DESCRIPTION OF THE DRAWINGS

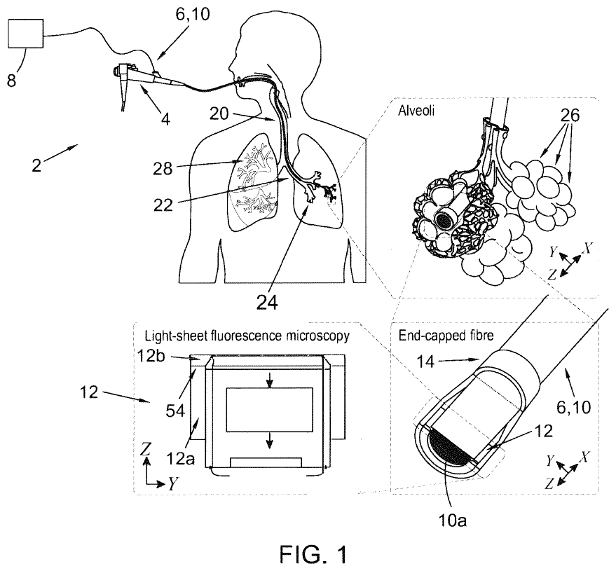

Referring initially to FIG. 1 there is shown a fluorescence endomicroscopy system generally designated 2 in use during fluorescence endomicroscopy of the lungs. The fluorescence endomicroscopy system 2 comprises a bronchoscope 4, a coherent fibre bundle (CFB) assembly generally designated 6, and instrumentation 8 connected to a proximal end of the CFB assembly 6. The coherent fibre bundle (CFB) assembly comprises a polymer CFB 10 comprising, or formed from, polymethyl methacrylate (PMMA), a generally annular end cap 12 attached to a distal end of the CFB 10, and an outer protective sleeve in the form of some heat-shrink tubing 14 around the end cap 12. As will be described in detail below, the CFB assembly 6 is inserted into one of the alveoli using the bronchoscope 4 via the trachea 20, the primary bronchus 22 and one of the bronchioles 24 and the CFB assembly 6 is used for selective plane illumination microscopy (SPIM) of a sample or material to be imaged in the form of tissue of the alveolar sacs 26 of a lung 28.

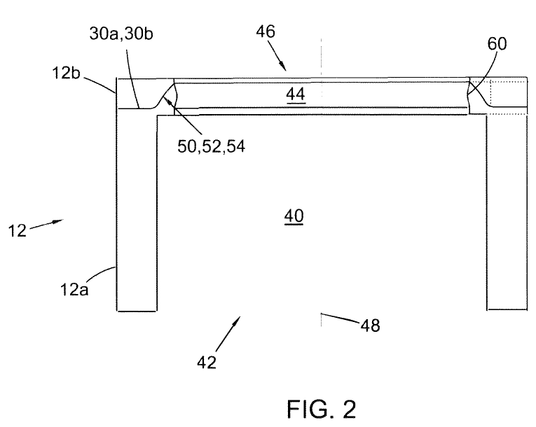

As shown in FIG. 2, the end cap 12 comprises a generally annular first part 12a and a generally annular second part 12b, wherein the first and second parts 12a, 12b are aligned co-axially along a longitudinal axis 48 and the first and second parts 12a, 12b comprise one or more complementary alignment features for aligning the first and second parts 12a, 12b relative to one another. Specifically, an upper surface 30a of the first part 12a and a lower surface 30b of the second part 12b have complementary profiles so that when the lower surface 30b of the second part 12b and the upper surface 30a of the first part 12a are brought into engagement, the features of the first and second parts 12a, 12b are aligned.

The end cap 12 defines a passageway which extends from a rear side 42 of the end cap 12 to a front side 46 of the end cap 12. The first part 12a of the end cap 12 comprises one or more CFB alignment features in the form of a rear space for receiving the distal end of the CFB 10, wherein the rear space is defined by a wider diameter rear section 40 of the passageway which extends from the rear side 42 of the first part 12a. The end cap 12 further comprises a sample space for receiving a sample or material to be imaged, wherein the sample space is defined by a narrower diameter front section 44 of the passageway which extends from the front side 46 of the end cap 12. Consequently, when the distal end of the CFB 10 is inserted into the wider diameter rear section 40 of the passageway, the distal end face 10a of the CFB 10 cannot protrude axially into the narrower diameter front section 44 of the passageway. The front and rear sections of the passageway 44, 40 are aligned co-axially along the longitudinal axis 48 of the end cap 12.

The first and second parts 12a, 12b together define a generally annular peripheral reflector 50 arranged around the front section 44 of the passageway. Specifically, the peripheral reflector 50 comprises a generally annular reflector surface 52 which is defined by the upper surface 30a of the first part 12a and which extends around the front section 44 of the passageway. As may be appreciated from FIG. 2, a normal to the reflector surface 52 extends along a direction having a radially outward component relative to the longitudinal axis 48 of the end cap 12. Furthermore, the reflector surface 52 has a curved profile when viewed on a longitudinal cross-section of the end cap 12 which includes the longitudinal axis 48 of the end cap 12, wherein the curved profile is outwardly convex relative to the longitudinal axis 48 of the end cap 12. Moreover, the lower surface 30b of the second part 12a of the end cap 12 comprises a reflective material or coating in the form of a silver coating 54 so that, when the lower surface 30b of the second part 12b and the upper surface 30a of the first part 12a are brought into engagement, the silver coating 54 covers the reflector surface 52.

The first part 12a of the end cap 12 further comprises a generally annular peripheral lens 60 located radially between the peripheral reflector 50 and the front section 44 of the passageway relative to the longitudinal axis 48. As shown in FIG. 2, the peripheral lens 60 at least partially defines the front section 44 of the passageway. Moreover, as may be appreciated from FIG. 2, the peripheral lens 60 defines a lens profile which is inwardly convex relative to the longitudinal axis 48 of the end cap 12.

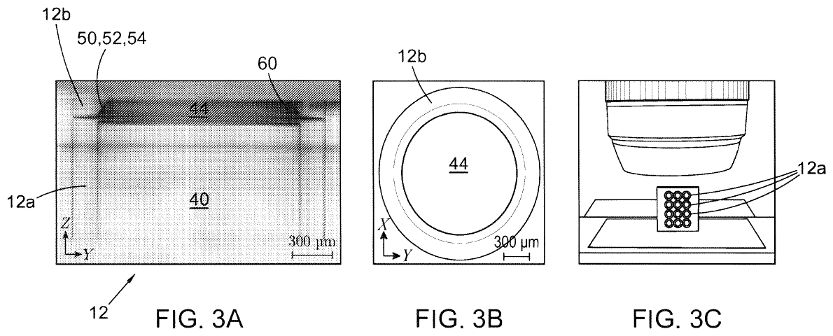

FIG. 3A is a side view microscope image of a region of a fused silica substrate and FIG. 3B is a plan view microscope image of the same region of the fused silica substrate after femtosecond laser inscription of the fused silica substrate so as to define the geometry of the end cap 12, but before chemical etching of the inscribed regions of the fused silica substrate. FIG. 3C is an image of a 10×10×1 mm substrate of fused silica in which twelve end cap parts 12a are inscribed.

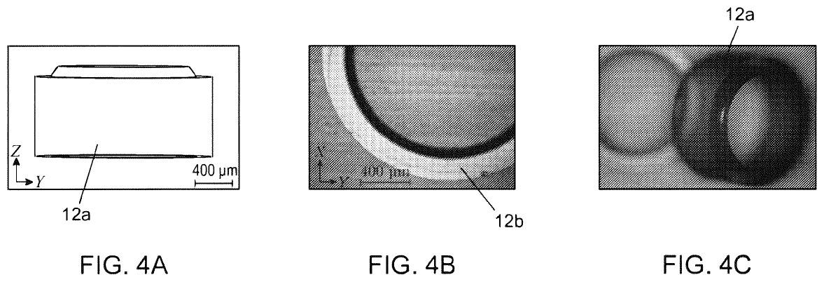

FIG. 4A is a side view microscope image of the first part 12a of the end cap 12 and FIG. 4B is a plan view microscope image of the second part 12b of the end cap 12 after a KOH etching process. FIG. 4C is a microscope image of the first part 12a of the end cap 12 in a slightly oblique position.

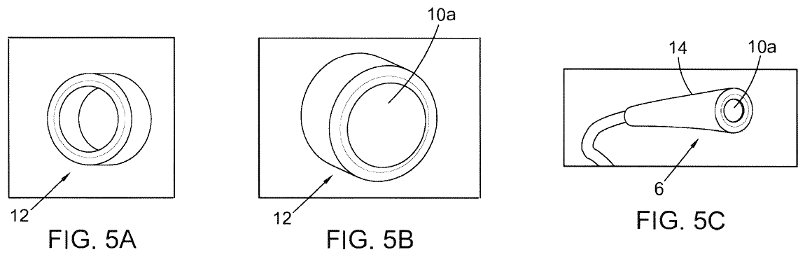

FIG. 5A is an image of the first and second parts 12a, 12b glued together to form the end cap 12. FIG. 5B is an image of the end cap 12 bonded to the PMMA CFB 10 and showing a distal end face 10a of the CFB 10. FIG. 5C is an image of a distal end of the CFB assembly 6 showing the distal end face 10a of the CFB 10 and the protective heat-shrink tubing 14 around the fused silica cap 12.

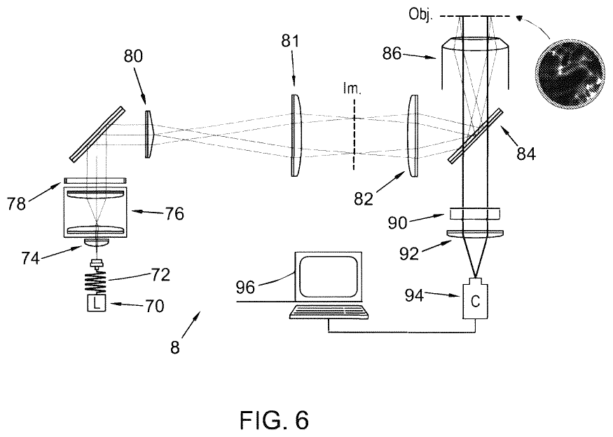

FIG. 6 is a schematic of the proximal end instrumentation 8 which takes the form of an epifluorescence microscope comprising a laser 70, a single-mode optical fibre patchcord 72, a collimating lens 74, a beam expander 76, and an excitation filter 78. The epifluorescence microscope further comprises an axicon lens 80, a first relay lens 81, a second relay lens 82, a dichroic mirror 84 and an objective lens 86. The epifluorescence microscope also comprises an emission filter 90, a focussing lens 92, an image sensor 94, and a computer 96. The image sensor 94 and the computer 96 are configured for communication.

In use, the proximal end instrumentation 8 provides ring excitation of the sample or material in the front section 44 of the passageway of the end cap 12 and retrieves the fluorescence images of the sample or material in the front section 44 of the passageway. Specifically, light from the laser 70 is transmitted through the single-mode optical fibre patchcord 72 and is collimated by the collimating lens 74. The collimated beam is then expanded by the beam expander 76 and passes through the excitation filter 78. The axicon lens 80 converts the collimated beam into a ring-shaped beam. An image plane of the ring-shaped beam is then created by the first relay lens 81. This image plane is relay-imaged onto the proximal end of the CFB 10 in the focal plane of the objective lens 86 using the second relay lens 82 and the objective lens 86 via the dichroic mirror 84. The axicon lens 80 and relay lenses 81, 82 are configured so as to create a predetermined ring of illumination on the proximal end of the CFB 10. Thus, only the outer optical cores of the CFB 10, which are capped at the distal end of the CFB 10 by the peripheral reflector 50, are illuminated with the excitation light from the laser 70. Moreover, the end cap 12 is configured so that the peripheral reflector 50 re-directs the excitation light output from the outer optical cores of the CFB 10 so that the re-directed excitation light propagates at least part way across the front section 44 of the passageway in front of the distal end face 10a of the CFB 10 for the excitation of the sample or material in the front section 44 of the passageway and the generation of fluorescence therein and so that at least a portion of the fluorescence is coupled into a plurality of inner optical cores of the CFB 10. The peripheral lens 60 concentrates or focuses the re-directed excitation light as the re-directed excitation light propagates at least part way across the front section 44 of the passageway in front of the end face 10a of the CFB 10 towards the longitudinal axis 48 of the end cap 12 so as to form a sheet of excitation light in front of the end face 10a of the CFB 10.

The plurality of inner optical cores of the CFB 10 transmit an image of the fluorescence emitted by the sample or material in the front section 44 of the passageway back to the proximal end of the CFB 10 and the image of the fluorescence emitted by the sample or material is imaged onto the image sensor 94 via the objective lens 86, the dichroic mirror 84, the emission filter 90, and the focussing lens 92.

Use of the end cap 12 means that the inner optical cores of the CFB 10 are not excited by the excitation light thereby avoiding, or at least partially suppressing, the generation of any auto-fluorescence background in the inner optical cores of the polymer CFB 10. This may improve the quality and/or contrast of the image of the sample. Installing the end cap 12 on the distal end of the CFB 10 may also enable SPIM so that only a region of the sample or material in the front section 44 of the passageway which is in close proximity to the end of the CFB 10, and which is therefore in-focus, is excited. Thus, use of the end cap 12 may avoid, or at least partially suppress, the generation in the sample or material of any out-of-focus fluorescence background.

The end cap 12 is also suitable for use with a single CFB 10 avoiding any need for any additional optical fibres. The end cap 12 also avoids any requirement for the use of additional optical components such as one or more GRIN lenses and/or prisms at the distal end of the CFB 10. For all of these reasons, use of the end cap 12 enables a CFB assembly 6 with a reduced footprint or volume compared with prior art fibre optic SPIM systems. In particular, when the end cap 12 is installed on the distal end of the CFB 10, the resulting assembly may have a reduced diameter relative to known CFB systems for SPIM. Use of the end cap 12 may also provide a larger field of view than known CFB systems for SPIM. These characteristics may make the end cap 12 advantageous for clinical use cases.

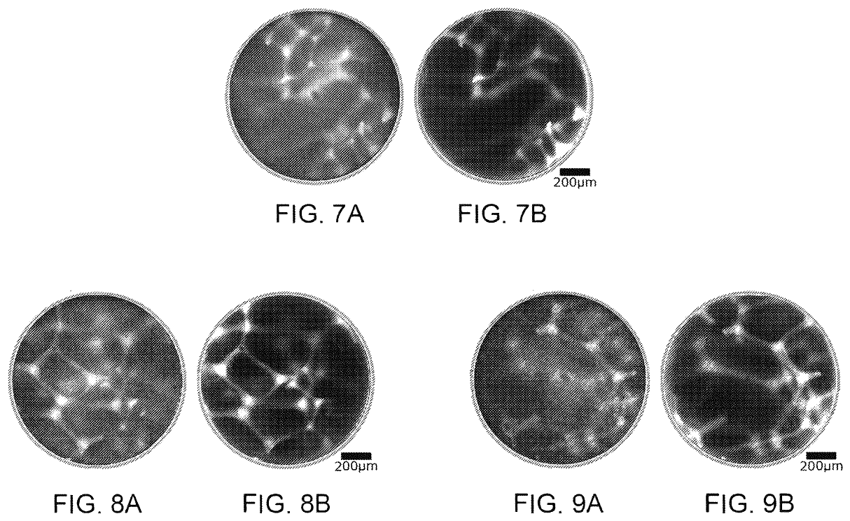

FIGS. 7A and 7B show images obtained of a first tissue phantom without and with selective plan illumination respectively, wherein the image of FIG. 7B was obtained using the CFB assembly 6 in combination with the proximal end instrumentation 8 of FIG. 6. Similarly, FIGS. 8A and 8B show images obtained of a second tissue phantom without and with selective plan illumination respectively, wherein the image of FIG. 8B was obtained using the CFB assembly 6 in combination with the proximal end instrumentation 8 of FIG. 6, and FIGS. 9A and 9B show images obtained of a third tissue phantom without and with selective plan illumination respectively, wherein the image of FIG. 9B was obtained using the CFB assembly 6 in combination with the proximal end instrumentation 8 of FIG. 6. As may be appreciated by comparing FIGS. 7B, 8B and 9B with FIGS. 7A, 8A and 9A respectively, use of the CFB assembly 6 including the end cap 12 and the proximal end instrumentation 8 of FIG. 6 for SPIM results in improved image quality and/or improved image contrast.

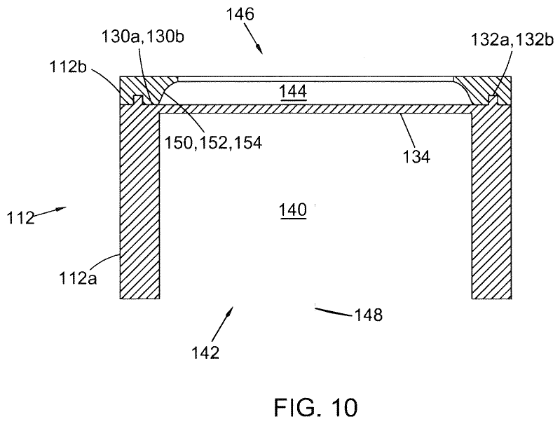

Referring now to FIG. 10, there is shown an alternative end cap 112 for use with the CFB 10. The alternative end cap 112 comprises a generally cylindrical first part 112a and a generally annular second part 112b, wherein the first and second parts 112a, 112b are aligned co-axially along a longitudinal axis 148 and the first and second parts 12a, 12b comprise one or more complementary alignment features for aligning the first and second parts 112a, 112b relative to one another. Specifically, an upper surface 130a of the first part 112a defines a generally annular ridge 132a and a lower surface 130b of the second part 112b defines a generally annular groove 132b so that when the groove 132b of the second part 112b and the ridge 132a of the first part 112a are in inter-engagement, the features of the first and second parts 112a, 112b are aligned.

The first part 112a of the end cap 112 comprises one or more CFB alignment features in the form of a rear space in the form of a rear recess 140 for receiving the distal end of the CFB 110, wherein the rear recess 140 extends from a rear side 142 of the first part 112a. The end cap 112 further comprises a sample space in the form of a front recess 144 for receiving a sample or material to be imaged, wherein the front recess 144 extends from a front side 146 of the end cap 112. The front and rear recesses 144, 140 are aligned co-axially along the longitudinal axis 148 of the end cap 112.

The second part 112b defines a generally annular peripheral reflector 150 arranged around the front recess 144. Specifically, the peripheral reflector 150 comprises a generally annular reflector surface 152 which is defined by the lower surface 130b of the second part 112b and which extends around the front recess 144. As may be appreciated from FIG. 10, a normal to the reflector surface 152 extends along a direction having a radially inward component relative to the longitudinal axis 148 of the end cap 112. Furthermore, the reflector surface 152 has a curved profile when viewed on a longitudinal cross-section of the end cap 112 which includes the longitudinal axis 148 of the end cap 112, wherein the curved profile is inwardly concave relative to the longitudinal axis 148 of the end cap 112. Moreover, the lower surface 130b of the second part 112a of the end cap 112 comprises a reflective material or coating in the form of a silver coating 154 which is formed or disposed on the reflector surface 152.

The first part 112a further comprises an intervening portion 134 which is configured to extend between the front recess 144 and an end face of the CFB at the distal end of the CFB 10 when the end cap 112 is aligned relative to the distal end of the CFB 10. Specifically, the intervening portion 134 extends between the front recess 144 and the rear recess 140. In use, the intervening portion 134 separates the end face of the CFB 10 from the sample or material in the front recess 144. The intervening portion 134 is configured to transmit at least a portion of the excitation light from the plurality of outer optical cores of the CFB 10 towards the peripheral reflector 150. The intervening portion 134 is also configured to transmit at least a portion of the fluorescence to the plurality of inner optical cores of the CFB 10 when the end cap 112 is aligned relative to the distal end of the CFB 10.

The curved profile of the reflector surface 152 is designed to re-direct and focus excitation light output from the plurality of outer optical cores of the CFB so that the re-directed excitation light forms a sheet of light which propagates at least part way across the front recess 144 in front of an end face of the CFB 10 for the excitation of the sample or material in the front recess 144 and the generation of fluorescence therein. At least a portion of the fluorescence is coupled into a plurality of inner optical cores of the CFB 10.

One of ordinary skill in the art will also understand that various modifications are possible to any of the end caps and end caps described above. For example, rather than being generally annular, the peripheral reflector 50 may extend only part way around the periphery of the front section 44 of the passageway. Similarly, rather than being generally annular, the peripheral reflector 150 may extend only part way around the periphery of the front recess 144.

Rather than being generally annular, the peripheral lens 60 may extend only part way around the front section 44 of the passageway.

Rather than being formed as two separate parts which are subsequently engaged and attached, the end cap may be unitary. For example, a reflective coating may be applied to the reflector surface 52 of the first part 12a of the end cap 12 thereby avoiding any need for the second part 12b.

In a variant of the end cap 12 of FIG. 2, the end cap may have an intervening portion which is configured to extend across the passageway in front of an end face of the CFB 10 at the distal end of the CFB 10 when the end cap 12 is aligned relative to the distal end of the CFB 10 so as to define a front recess for receiving a sample or material to be imaged on a front side of the intervening portion and a rear recess for receiving the distal end of the CFB 10.

In a variant of the end cap 112 of FIG. 10, the end cap may not have an intervening portion like the intervening portion 134 extending between the front and rear recesses 144, 140. Instead, the end cap may define a passageway which extends from the rear side of the end cap to the front side of the end cap. The passageway may include a rear space in the form of a wider diameter rear section which extends from the rear side of the end cap and which is configured to receive the distal end of the CFB 10. The passageway may include a sample space in the form of a narrower diameter front section which extends from the front side of the end cap and which is configured to receive a sample or material to be imaged. Consequently, when the distal end of the CFB 10 is inserted into the wider diameter rear section of the passageway, the distal end face 10a of the CFB 10 cannot protrude axially into the narrower diameter front section of the passageway. The end cap may be generally annular.

Although each of the end caps 12, 112 have been described above as being configured for use with a PMMA CFB, the end cap may be configured for use with a coherent fibre bundle (CFB) which comprises, or is formed from, a polymer material of any kind or the end cap may be configured for use with a CFB which comprises, or is formed from, a glass material.

The end cap 12 may be configured so that, when the end cap 12 is aligned relative to the distal end of the CFB 10, the peripheral reflector 50 re-directs excitation light output from the plurality of outer optical cores of the CFB 10 so that the re-directed excitation light propagates at least part way across the front section 44 of the passageway in front of the end face of the CFB 10 for the excitation of the sample or material in the front section 44 of the passageway and the generation of Raman scattered light therein and so that at least a portion of the Raman scattered light is coupled into the plurality of inner optical cores of the CFB 10. Similarly, the end cap 112 may be configured so that, when the end cap 112 is aligned relative to the distal end of the CFB 10, the peripheral reflector 150 re-directs excitation light output from the plurality of outer optical cores of the CFB 10 so that the re-directed excitation light propagates at least part way across the front recess 144 in front of the end face of the CFB 10 for the excitation of the sample or material in the front recess 144 and the generation of Raman scattered light therein and so that at least a portion of the Raman scattered light is coupled into the plurality of inner optical cores of the CFB 10. Such end caps may be used for SPIM imaging of the Raman scattered light.

Although the disclosure has been described in terms of preferred embodiments as set forth above, it should be understood that these embodiments are illustrative only and that the claims are not limited to those embodiments. Those skilled in the art will be able to make modifications and alternatives to the described embodiments in view of the disclosure which are contemplated as falling within the scope of the appended claims. Each feature disclosed or illustrated in the present specification may be incorporated in any embodiment, whether alone or in any appropriate combination with any other feature disclosed or illustrated herein. In particular, one of ordinary skill in the art will understand that one or more of the features of the embodiments of the present disclosure described above with reference to the drawings may produce effects or provide advantages when used in isolation from one or more of the other features of the embodiments of the present disclosure and that different combinations of the features are possible other than the specific combinations of the features of the embodiments of the present disclosure described above.

The skilled person will understand that in the preceding description and appended claims, positional terms such as ‘above’, ‘along’, ‘side’, etc. are made with reference to conceptual illustrations, such as those shown in the appended drawings. These terms are used for ease of reference but are not intended to be of limiting nature. These terms are therefore to be understood as referring to an object when in an orientation as shown in the accompanying drawings.

Use of the term “comprising” when used in relation to a feature of an embodiment of the present disclosure does not exclude other features or steps. Use of the term “a” or “an” when used in relation to a feature of an embodiment of the present disclosure does not exclude the possibility that the embodiment may include a plurality of such features.

The use of reference signs in the claims should not be construed as limiting the scope of the claims.

1. An end cap for a coherent fibre bundle (CFB) for enabling selective plane illumination microscopy (SPIM), the end cap comprising:
one or more CFB alignment features for aligning the end cap relative to a distal end of a CFB;
a sample space for receiving a sample or material to be imaged, wherein the sample space extends from a front side of the end cap; and
a peripheral reflector arranged at least part way around the sample space,
wherein the end cap is configured so that, when the end cap is aligned relative to the distal end of the CFB, the peripheral reflector re-directs excitation light output from a plurality of outer optical cores of the CFB so that the re-directed excitation light propagates at least part way across the sample space in front of an end face of the CFB for the excitation of the sample or material in the sample space and the generation of fluorescence therein and so that at least a portion of the fluorescence is coupled into a plurality of inner optical cores of the CFB.

2. The end cap as claimed in claim 1, wherein the peripheral reflector is annular or generally annular.

3. The end cap as claimed in claim 1, wherein the peripheral reflector defines a reflector surface which extends at least part way around the sample space.

4. The end cap as claimed in claim 3, wherein the end cap defines a longitudinal axis for alignment with a longitudinal axis of the CFB and wherein a normal to the reflector surface extends along a direction having a radially outward component relative to the longitudinal axis of the end cap, wherein the reflector surface has a linear profile or a curved profile when viewed on a longitudinal cross-section of the end cap which includes the longitudinal axis of the end cap, and wherein the curved profile of the reflector surface is outwardly convex relative to the longitudinal axis of the end cap.

5. (canceled)

6. (canceled)

7. The end cap as claimed in claim 4, comprising a peripheral lens arranged at least part way around the sample space and located radially between the peripheral reflector and the sample space relative to the longitudinal axis, wherein the peripheral lens is configured to concentrate or focus the re-directed excitation light as the re-directed excitation light propagates at least part way across the sample space in front of the end face of the CFB towards the longitudinal axis of the end cap.

8. The end cap as claimed in claim 7, wherein the peripheral lens defines a lens profile which is inwardly convex relative to the longitudinal axis of the end cap.

9. The end cap as claimed in claim 7, wherein the peripheral lens at least partially defines the sample space.

10. The end cap as claimed in claim 7, wherein the peripheral lens is annular or generally annular.

11. The end cap as claimed in claim 3, wherein the end cap defines a longitudinal axis for alignment with a longitudinal axis of the CFB and wherein a normal to the reflector surface extends along a direction having a radially inward component relative to the longitudinal axis of the end cap, wherein the reflector surface has a linear profile or a curved profile when viewed on a longitudinal cross-section of the end cap which includes the longitudinal axis of the end cap, and wherein the curved profile of the reflector surface is inwardly concave relative to the longitudinal axis of the end cap.

12. (canceled)

13. (canceled)

14. The end cap as claimed in claim 3, wherein the peripheral reflector comprises a reflective material or coating which is formed on, or disposed on, the reflector surface or which covers the reflector surface.

15. The end cap as claimed in claim 1, wherein the one or more CFB alignment features comprise a rear space for receiving the distal end of the CFB, wherein the rear space extends from a rear side of the end cap.

16. The end cap as claimed in claim 1, comprising an intervening portion which is configured to extend between the sample space and an end face of the CFB at the distal end of the CFB when the end cap is aligned relative to the distal end of the CFB.

17. The end cap as claimed in claim 1, wherein the end cap is unitary.

18. The end cap as claimed in claim 1, wherein the end cap comprises first and second parts, wherein the first and second parts comprise one or more complementary alignment features for aligning the first and second parts relative to one another.

19. The end cap as claimed in claim 18, wherein:
the first part defines the one or more CFB alignment features, and wherein the first and second parts together define the peripheral reflector and, wherein the first part defines the one or more CFB alignment features and a reflector surface of the peripheral reflector, and the second part defines a reflective material or coating which covers the reflector surface when the first and second parts are aligned; or
the first part defines the one or more CFB alignment features and the second part defines the peripheral reflector.

20. (canceled)

21. The end cap as claimed in claim 1, wherein the end cap is configured for use with a coherent fibre bundle (CFB) which comprises, or is formed from, a polymer material such as PMMA or wherein the end cap is configured for use with a coherent fibre bundle (CFB) which comprises, or is formed from, a glass material.

22. The end cap as claimed in claim 1, wherein the end cap is configured so that, when the end cap is aligned relative to the distal end of the CFB, the peripheral reflector re-directs excitation light output from the plurality of outer optical cores of the CFB so that the re-directed excitation light propagates at least part way across the sample space in front of the end face of the CFB for the excitation of the sample or material in the sample space and the generation of Raman scattered light therein and so that at least a portion of the Raman scattered light is coupled into the plurality of inner optical cores of the CFB.

23. The end cap as claimed in claim 1, wherein the end cap comprises, or is formed from, fused-silica.

24. A coherent fibre bundle (CFB) assembly for SPIM, the CFB assembly comprising a coherent fibre bundle (CFB) and the end cap as claimed in claim 1 attached to a distal end of the CFB.

25. The coherent fibre bundle (CFB) assembly as claimed in claim 24, wherein the coherent fibre bundle (CFB) comprises, or is formed from, a polymer material such as PMMA or wherein the coherent fibre bundle (CFB) comprises, or is formed from, a glass material.
